(19) United States
(12) Patent Application Publication
(10) Pub. No.: US 2016/0206969 A1
(43) Pub. Date: Jul. 21, 2016

(54) Soap Bubble-Generating System

(71) Applicants: Yutaro KYONO, Kanagawa (JP); MOLCURE, INC., Tokyo (JP)

(72) Inventors: Yutaro Kyono, Tokyo (JP); Takuro Yonezawa, Tokyo (JP)

(21) Appl. No.: 14/917,268

(22) PCT Filed: Sep. 9, 2013
(86) PCT No.: PCT/JP2013/074287

§ 371 Date: Mar. 7, 2016

(51) Int. Cl. A63H 33/28; G10L 15/22; H04N 9/31; G06F 3/01; A61B 5/0476; A42B 1/00

(52) U.S. Cl. CPC A63H 33/28; A61B 5/0476; A42B 1/004; H04N 9/3173; G06F 3/015; G10L 15/22; G10L 2015/223

(57) ABSTRACT

To expand an information-presenting capacity of soap bubble, a soap bubble-generating system includes: a fragrance generating unit which generates a fragrance; a soap film generating unit which generates a soap film; and a soap bubble generating unit which sprays a fragrance-containing gas toward the soap film to generate a soap bubble in which the fragrance is enclosed.

### TECHNICAL FIELD

The present invention relates to a soap bubble-generating system.

### BACKGROUND ART

In recent years, a projection mapping system has been widely used. The projection mapping system is able to provide users with various visual impressions by projecting an image (video image) onto all kinds of objects in the real world. For example, Non-Patent Document 1 discloses a system which detects the position and size of each soap bubble floating in the air with a camera and projects an image only in the position using a projector.

### CITATION LIST

### Non-Patent Document

Non-Patent Document 1: Masahiro Nakamura and 4 others, “Mounting and application of bubble display system,” IPSJ Research Report, Information Processing Society of Japan, Dec. 17, 2005, Entertainment computing 2005 (125), pp. 33-40

### SUMMARY

### Technical Problem

Although the system disclosed in Non-Patent Document 1 is able to present information by projecting an image onto a soap bubble as described above, the image appeals only to the visual sense and it is desired to expand the information presenting capacity further.

Therefore, it is an object of the present invention to expand the information-presenting capacity of soap bubbles.

### Solution to Problem

According to one aspect of the present invention, there is provided a soap bubble-generating system including: a fragrance generating unit which generates a fragrance; a soap film generating unit which generates a soap film; and a soap bubble generating unit which sprays a gas containing the fragrance toward the soap film to generate a soap bubble in which the fragrance is enclosed.

In the present invention, the term “unit” does not simply mean a physical means, but also includes a case where a function of the “unit” is implemented by software. Furthermore, a function of one “unit” or device may be implemented by two or more physical means or devices, or functions of two or more “units” or devices may be implemented by one physical means or device.

### Advantageous Effects of Invention

According to the present invention, it is possible to expand the information-presenting capacity with soap bubbles.

### BRIEF DESCRIPTION OF DRAWINGS

FIG. 1 is a diagram illustrating the schematic configuration of a soap bubble-generating system which is one embodiment of the present invention.

FIG. 2 is a diagram illustrating an application example of the soap bubble-generating system.

### DESCRIPTION OF EMBODIMENTS

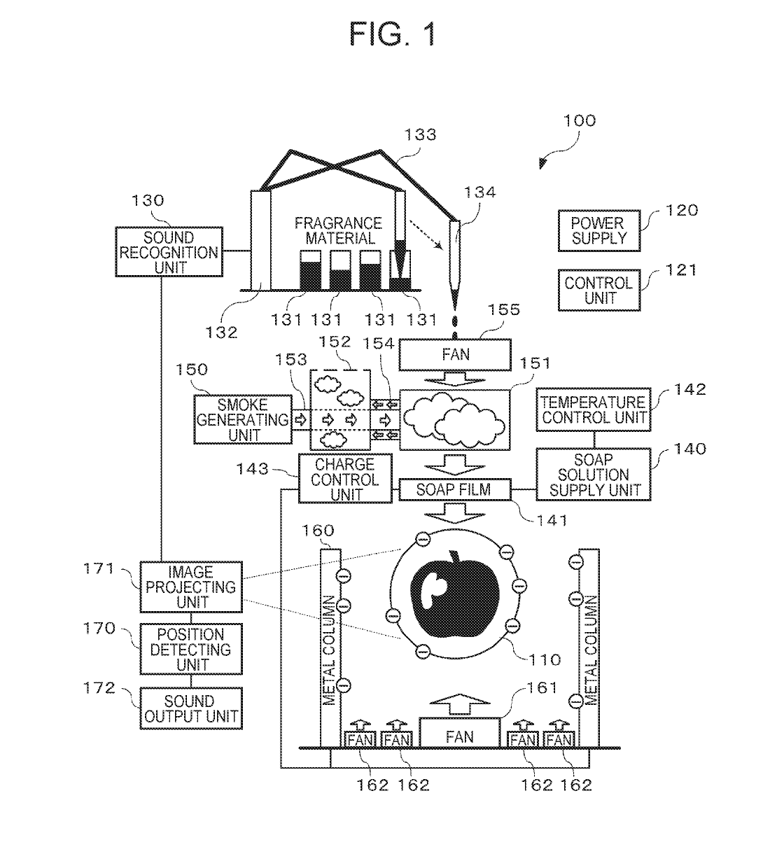

Hereinafter, one embodiment of the present invention will be described with reference to appended drawings. FIG. 1 is a diagram illustrating the schematic configuration of a soap bubble-generating system which is one embodiment of the present invention. The soap bubble-generating system 100 illustrated in FIG. 1 is able to generate a soap bubble 110 in which a fragrance is enclosed. Furthermore, the soap bubble-generating system 100 is able to project an image (video image) onto the soap bubble 110 and to control the position of the soap bubble 110.

The soap bubble-generating system 100 includes a power supply 120, a control unit 121, a sound recognition unit 130, a fragrance material storage unit 131, a fragrance control unit 132, a movable unit 133, a fragrance supply unit 134, a soap solution supply unit 140, a soap film generating unit 141, a temperature control unit 142, a charge control unit 143, a smoke generating unit 150, a reservoir unit 151, a buffer 152, paths 153 and 154, a fan 155, a metal column 160, fans 161 and 162, a position detecting unit 170, an image projecting unit 171, and a sound output unit 172.

The power supply 120 supplies power to respective units of the soap bubble-generating system 100. The power supply 120 is able to generate a voltage at a predetermined level from, for example, a commercial power supply. Furthermore, the power supply 120 may supply power to the respective units from a battery.

The control unit 121 controls the operations of the respective units of the soap bubble-generating system 100. The control unit 121 includes, for example, a processor and a memory and is able to implement various functions by executing programs stored in the memory by using a processor.

The sound recognition unit 130 is able to recognize a voice sound of a user of the soap bubble-generating system 100. In this embodiment, the sound recognition unit 130 is able to recognize sounds representing the types of fragrances such as, for example, “apple,” “rose,” and the like. The sound recognition unit 130 outputs a fragrance signal corresponding to the recognized type of fragrance.

The fragrance material storage unit 131 stores a fragrance material which is the cause of the fragrance. The fragrance material may be either liquid or powder. In this embodiment, a plurality of fragrances is able to be enclosed in the soap bubble 110. Therefore, a plurality of fragrance material storage units 131 is provided.

The fragrance control unit 132 controls the movable unit 133 and the fragrance supply unit 134 so that the fragrance specified by a user is generated on the basis of the fragrance signal received from the sound recognition unit 130. The movable unit 133 is, for example, a movable arm. Furthermore, the fragrance supply unit 134 is, for example, a pipette. The fragrance control unit 132 is able to fill the fragrance supply unit 134 with a predetermined amount of fragrance material by driving the movable unit 133 to move the tip of the fragrance supply unit 134 to the inside of an arbitrary fragrance material storage unit 131. Then, the fragrance control unit 132 is able to supply (drop) the fragrance material to the fan 155 by driving the movable unit 133 to move the fragrance supply unit 134 filled with the fragrance material to a position above the fan 155.

The soap solution supply unit 140 supplies soap solution for generating the soap bubble 110 to the soap film generating unit 141. The soap solution supply unit 140 is allowed to include, for example, a soap solution reservoir unit in which soap solution is retained and a movable unit for guiding the opening of the soap film generating unit 141 to the soap solution reservoir unit.

The soap film generating unit 141 has an (for example, a ring-shaped) opening and generates a soap film on the opening with the soap solution supplied from the soap film supply unit 140.

The temperature control unit 142 is able to control the temperature of the soap solution supplied from the soap solution supply unit 140 to a temperature at which the soap bubble 110 is not easily broken (for example, about 32° C.).

The charge control unit 143 is able to charge the soap bubble 110, for example, negatively. Specifically, the charge control unit 143 is able to charge the soap film generated by the soap film generating unit 141, for example, negatively. Moreover, the charge control unit 143 is able to charge the metal column 160, for example, negatively. Incidentally, the charge control unit 143 may be configured to charge the soap film or the metal column 160 positively. Furthermore, the charge control unit 143 may be configured to charge the soap bubble 110 by charging gas enclosed in the soap bubble 110, instead of charging the soap film.

The smoke generating unit 150 generates opaque smoke enclosed into the soap bubble 110. For example, the smoke generating unit 150 is able to generate smoke by inserting water-soluble smoke liquid into a heater by using a pump to evaporate the smoke liquid. The smoke generating unit 150 is able to send the generated smoke to the path 153.

The reservoir unit 151 retains the smoke generated by the smoke generating unit 150. The smoke generated by the smoke generating unit 150 is sent out to the reservoir unit 151 through the path 153. The smoke overflowing from the reservoir unit 151 is sent from the reservoir unit 151 to the buffer 152 through the path 154 provided separately from the path 153 and discharged from, for example, the top of the buffer 152.

The fan 155 is able to send an air flow to the soap film generated by the soap film generating unit 141 through the reservoir unit 151. Furthermore, the fan 155 is able to vaporize the fragrance material of the liquid supplied from the fragrance supply unit 134 and to send the vaporized fragrance material to the reservoir unit 151. Thereby, the gas and smoke including the fragrance are sprayed toward the soap film. Incidentally, the fan 155 may be a counter-rotating fan. Thus, the air flow sprayed toward the soap film is formed linearly, thereby making it more difficult for the soap film to be broken in generating the soap bubble 110.

The metal column 160 is provided to control the position of the soap bubble 110. In this embodiment, a plurality of metal columns 160 is provided. Each metal column 160 is charged to the same polarity as the soap bubble 110 (for example, to the negative polarity) by the charge control unit 143. Thereby, the soap bubble 110 and the metal column 160 repel each other and the position of each soap bubble 110 is controlled to a position between the plurality of metal columns 160. Incidentally, the metal columns 160 may be either fixed or movable to arbitrary positions by a user. If the metal columns 160 are movable, a part of each metal column 160 may be coated with an insulating material such as, for example, rubber in order to grip the metal column 160. Furthermore, the metal column 160 is not limited to a column-shaped one. Instead, it is possible to use chargeable one having an arbitrary shape, thereby enabling the position of the soap bubble 110 to be controlled. Moreover, it is possible to use a metal column 160 charged to a polarity opposite to that of the soap bubble 110 to pull the soap bubble 110.

The fans 161 and 162 are provided to control the position of the soap bubble 110. In this embodiment, the fan 161 is arranged substantially directly below the opening of the soap film generating unit 141 and the plurality of fans 162 are arranged around the fan 161. It is possible to control the vertical position of the soap bubble 110 by mainly adjusting the air blown from the fan 161 and to control the horizontal position of the soap bubble 110 by mainly adjusting the air blown from the fans 162. In this manner, the soap bubble 110 can be controlled to various positions by adjusting the air blown from the fans 161 and 162. Moreover, the soap bubble 110 may be controllable to various positions by changing the positions, heights, and orientations for installing the fans 161 and 162 or by adjusting the direction, volume, or the like of the air flow of the fans 161 and 162.

The position detecting unit 170 detects the position of the soap bubble 110. The position detecting unit 170 is allowed to include a camera, for example, capable of measuring a depth. Then, the position detecting unit 170 is able to detect the position of the soap bubble 110, whose distance from the camera is located within a predetermined range in a range captured by the camera, on the basis of the captured image and the depth information. Furthermore, the position detecting unit 170 is also able to detect that the soap bubble 110 is broken by a change in the captured image.

The image projecting unit 171 projects an image (video image) onto the soap bubble 110 on the basis of the detection result of the position detecting unit 170. Since the position detecting unit 170 continues to detect the position of the soap bubble 110, the image projecting unit 171 is able to project an image onto the soap bubble 110 in accordance with the movement of the soap bubble 110. Incidentally, the image projecting unit 171 is able to project an image related to the fragrance enclosed in the soap bubble 110 onto the soap bubble 110 on the basis of the sound recognition result obtained by the sound recognition unit 130. For example, in the case where the fragrance of “apple” is enclosed in the soap bubble 110, the image projecting unit 171 may project an image of “apple” onto the soap bubble 110. The fragrance and the image do not need to have a direct relevance such as a fragrance of “apple” and an image of “apple.” For example, an image of a landscape or the like recalled from the fragrance may be projected onto the soap bubble 110.

The sound output unit 172 is able to output a sound effect when the soap bubble 110 is broken on the basis of the detection result of the position detecting unit 170. Furthermore, the sound output unit 172 is able to output various sound effects in the soap bubble-generating system 100. For example, the sound output unit 172 is able to output a sound indicating a sound recognition result obtained by the sound recognition unit 130 or to output a sound indicating the operation state of each unit.

In the soap bubble-generating system 100 described above, the fragrance material storage unit 131, the fragrance control unit 132, the movable unit 133, and the fragrance supply unit 134 constitute a fragrance generating unit. Moreover, the reservoir unit 151, the buffer 152, the path 153, the path 154, and the fan 155 constitute a soap bubble generating unit. Furthermore, the fan 155 constitutes a fragrance sending unit and a spraying unit. In addition, the buffer 152, the path 153, and the path 154 constitute a smoke sending unit. Furthermore, the metal column 160 constitutes a position control unit. Moreover, the sound recognition unit 130 constitutes a fragrance signal output unit.

The following describes a flow of generating a soap bubble in the soap bubble-generating system 100. When a user utters with a sound (for example, “apple”) specifying the type of fragrance, the sound recognition unit 130 recognizes the sound. The fragrance control unit 132 controls the movable unit 133 and the fragrance control unit 134 on the basis of the recognition result of the sound recognition unit 130 and fills the fragrance supply unit 134 with the fragrance material for generating the fragrance specified by the user.

The soap film generating unit 141 generates a soap film on the opening of the soap film generating unit 141 by using the soap solution supplied from the soap solution supply unit 140. Furthermore, the charge control unit 143 charges the generated soap film and the metal column 160, for example, negatively.

The smoke generating unit 150 generates smoke and sends out the smoke to the reservoir unit 151 through the path 153. At this time, the smoke overflowing from the reservoir unit 151 is sent out to the buffer 152 through the path 154. Incidentally, the openings for passing an air flow from the fan 155 may be closed by shutters or the like until the air flow is sent out from the fan 155, where the openings are provided at the top and bottom of the reservoir unit 151.

After the soap film is generated and smoke is retained in the reservoir unit 151, the fan 155 sends out an air flow toward the soap film from the reservoir unit 151. At this time, the fragrance supply unit 134 supplies the fan 155 with the fragrance material. Thereby, the soap bubble 110 in which gas and smoke including the fragrance are enclosed is generated. Moreover, the fan 161 sends out an upward air flow.

Upon the generation of the soap bubble 110, the position detecting unit 170 detects the position of the soap bubble 110. Then, the image projecting unit 171 projects an image onto the soap bubble 110 on the basis of the detected position.

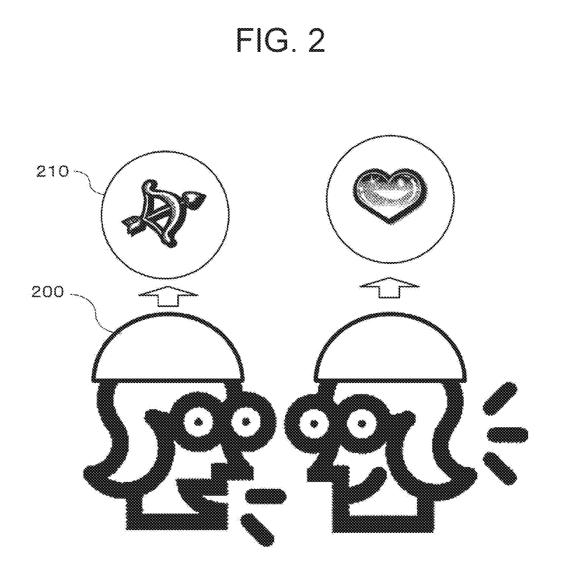

The following describes an application example of the soap bubble-generating system 100. FIG. 2 illustrates an application of the soap bubble-generating system 100 illustrated in FIG. 1. A soap bubble-generating system 200 illustrated in FIG. 2 is configured to be mountable on a human head. The soap bubble-generating system 200 includes inside the elements equivalent to those of the soap bubble-generating system 100 illustrated in FIG. 1. Incidentally, the soap bubble-generating system 200 includes a brain wave detector, instead of the sound recognition unit 130.

The soap bubble-generating system 200 generates a soap bubble 210 in which fragrance corresponding to a detection result of the brain wave detector is enclosed. For example, in the case where one person wearing the soap bubble-generating system 200 is supposed to be biased in favor of the other person, the soap bubble-generating system 200 may generate a soap bubble 210 in which sweet fragrance is enclosed. Moreover, the soap bubble-generating system 200 is able to project an image related to fragrance onto the soap bubble 210 similarly to the soap bubble-generating system 100.

One embodiment of the present invention has been described hereinabove. According to this embodiment, the present invention is able to generate a soap bubble in which fragrance is enclosed. This enables a soap bubble not only to be captured visually, but also to appeal to the human sense of smell by the fragrance released when the soap bubble is broken. In other words, the information-presenting capacity with a soap bubble can be expanded.

Furthermore, according to this embodiment, an image is able to be projected onto a soap bubble. This enables visual information presentation by using an image in addition to the olfactory information presentation by using fragrance.

Furthermore, according to this embodiment, an image projected onto a soap bubble is able to be associated with fragrance thereof. This enables further enhancement of the information-presenting capacity due to a synergetic effect between the image and the fragrance. For example, a combination of the image and the fragrance makes it easier to recall a memory related to the image.

Furthermore, according to this embodiment, opaque smoke can be enclosed in a soap bubble. This enables enhancement of visibility of the soap bubble itself. In addition, the smoke enclosed in the soap bubble makes it easier to see the image projected onto the soap bubble.

Furthermore, according to this embodiment, smoke overflowing from the reservoir unit is able to be sent out to a place (for example, a buffer) other than the reservoir unit. Thereby, the amount of smoke enclosed in the soap bubble can be adjusted so as to be appropriate.

Moreover, according to this embodiment, the position of a soap bubble can be controlled by charging the soap bubble. This enables the soap bubble to stay in a predetermined position or to move to an arbitrary position where a fragrance performance is desired to be provided.

Moreover, according to this embodiment, the type of fragrance can be specified by a user input (for example, sound recognition or brain wave detection). This makes it possible to enjoy various types of fragrance. Particularly, with the generation of a soap bubble in which fragrance corresponding to the detection result of a brain wave is enclosed, it is possible to provide epoch-making information presentation which has never been known such that what a person is thinking is delivered by means of fragrance.

This embodiment is intended to facilitate the understanding of the present invention, but is not intended to limit the present invention in interpretation. The present invention is able to be modified or improved without departing from the spirit of the invention and includes equivalents thereof.

### REFERENCE SIGNS LIST

100 Soap bubble-generating system
110 Soap bubble
120 Power supply
121 Control unit
130 Sound recognition unit
131 Fragrance material storage unit
132 Fragrance control unit
133 Movable unit
134 Fragrance supply unit
140 Soap solution supply unit
141 Soap film generating unit
142 Temperature control unit
143 Charge control unit
150 Smoke generating unit
151 Reservoir unit
152 Buffer
153, 154 Path
155, 161, 162 Fan
160 Metal column
170 Position detecting unit
171 Image projecting unit
172 Sound output unit

1. A soap bubble-generating system comprising:
a fragrance generating unit which generates a fragrance;
a soap film generating unit which generates a soap film; and
a soap bubble generating unit which sprays a gas containing the fragrance toward the soap film to generate a soap bubble in which the fragrance is enclosed.

2. The soap bubble-generating system according to claim 1, further comprising:
a position detecting unit which detects a position of the soap bubble; and
an image projecting unit which projects an image onto the soap bubble based on the detected position.

3. The soap bubble-generating system according to claim 2, wherein the image projecting unit projects an image related to the fragrance onto the soap bubble.

4. The soap bubble-generating system according to claim 1, further comprising a smoke generating unit which generates opaque smoke, wherein the soap bubble generating unit sprays the gas containing the fragrance and the smoke toward the soap film to generate a soap bubble in which the fragrance and the smoke are enclosed.

5. The soap bubble-generating system according to claim 4, wherein the soap bubble generating unit includes:
a reservoir unit which retains a gas;
a fragrance sending unit which sends out a gas containing the fragrance to the reservoir unit;
a smoke sending unit which sends out the smoke to the reservoir unit; and
a spraying unit which sprays the gas containing the fragrance and the smoke retained in the reservoir unit toward the soap film.

6. The soap bubble-generating system according to claim 5, wherein the smoke sending unit sends out the smoke overflowing from the reservoir unit to a place other than the reservoir unit.

7. The soap bubble-generating system according to claim 6, wherein the smoke sending unit, which includes first and second paths connected to the reservoir unit, sends out the smoke to the reservoir unit through the first path and sends out the smoke overflowing from the reservoir unit to a place other than the reservoir unit through the second path.

8. The soap bubble-generating system according to claim 1, further comprising:
a charge control unit which charges the soap bubble positively or negatively; and
a position control unit which is charged positively or negatively and controls the position of the charged soap bubble.

9. The soap bubble-generating system according to claim 1, further comprising a fragrance signal output unit which outputs a fragrance signal specifying a type of fragrance according to a user input, wherein the fragrance generating unit generates the fragrance of a type corresponding to the fragrance signal.

10. The soap bubble-generating system according to claim 9, wherein the fragrance signal output unit includes a sound recognition unit which recognizes a sound as a user input and outputs the fragrance signal based on a result of recognizing the sound.

11. The soap bubble-generating system according to claim 9, wherein the fragrance signal output unit includes a brain wave detector which detects a brain wave as a user input and outputs the fragrance signal based on a result of detecting the brain wave.

12. The soap bubble-generating system according to claim 11, wherein the soap bubble-generating system is configured so as to be mountable on a human head.
